(19) United States
(12) Patent Application Publication
(10) Pub. No.: US 2023/0407240 A1
(43) Pub. Date: Dec. 21, 2023

(54) MINIMAL FOOTPRINT HIGH DENSITY FERMENTATION OF PLANT BYPRODUCTS

(71) Applicant: KULA BIO, INC., Natick, MA (US)

(72) Inventors: Frederic KENDIRGI, Natick, MA (US); Sung-Yong Harrison YOON, Natick, MA (US)

(73) Assignee: KULA BIO, INC., Natick, MA (US)

(21) Appl. No.: 18/033,514

(22) PCT Filed: Oct. 25, 2021
(86) PCT No.: PCT/US2021/056502

§ 371 Date: Apr. 24, 2023

Related U.S. Application Data
(60) Provisional application No. 63/105,276, filed on Oct. 24, 2020.

(51) Int. Cl. C12N 1/20; C12M 1/00; C12M 1/34; C12M 1/21; C12M 1/26

(52) U.S. Cl. CPC C12N 1/20; C12M 23/58; C12M 41/26; C12M 33/14; C12M 41/36; C12M 41/02; C12M 41/34

(57) ABSTRACT

The present disclosure provides a method of preparing a high density culture of bacteria. The method provides a bioreactor comprising a fermentation tank, a perfusion system, a medium, and a cell separator; preparing an inoculum comprising a bacteria consortia with at least two bacteria species; inoculating the fermentation tank with the inoculum to form a fermentation culture; and growing the bacteria consortia until it has reached a target density of at least about 10¹² CFU/mL.

### CROSS REFERENCE TO RELATED APPLICATIONS

The present application claims the priority benefit of U.S. Provisional Application No. 63/105,276, filed Oct. 24, 2020, which is hereby incorporated by reference in its entirety.

### FIELD OF DISCLOSURE

The present disclosure pertains to bacteria cultivation. Certain aspects of the disclosure relate to methods of preparing a high density culture of a bacteria consortia with at least two bacteria species.

### BACKGROUND

Traditional fermentation scale-ups (i.e., a volumetric scale-up) are not suited for co-cultivation of microbial consortia. Microbial communities form complex heterogeneous systems with diverse metabolic activities and ecological dependencies. The benefits of microbial communities in agriculture are well known and documented, as evidenced by new Agriculture industry and farming practices. Microbe-microbe interactions (both intraspecies and interspecies) within established complex communities have yet to be fully deciphered. Even less is known about the biological and physical mechanisms that lead to the establishment and maintenance of stable bacteria consortia in nature—let alone in artificial environments such as fermentation tanks.

Great technological advancements have been achieved in the development of synthetic microbial consortia prototypes, including elucidation of optimal growth conditions to achieve products with the desired effects. This is evidenced by the high number of both academic and industrial research activities with seemingly great success, as judged by the plethora of scientific publications on this subject matter and the numerous products already on the market and in various stages of development.

However, the technology for scaling-up these prototypes has not followed suit. Volumetric seed train type scale-up processes are being used and are continuously developed and optimized. However, this technology possesses significant challenges.

From a biological standpoint, little is known about how the microbes interact with one another to form a stable consortium at the onset of their introduction to one another. Although reproducible in first generation amplifications, there is no guarantee that the microbial equilibrium achieved at the end of the first amplification will be maintained as the consortium is further amplified in a stepwise process to larger volumes—the definition of volumetric scale up processes. This has led many to shy away from co-cultivation of microbes in favor of producing each strain individually as mono-cultures and mixing them together post-amplification to produce the final product. Although the microbial composition in the final product derived for large-scale mon-cultures are adjusted to reflect that which resulted from the small-scale co-cultivation prototype, the benefits from co-cultivation of microbes is lost. Indeed, co-cultures have many advantages compared to mono-cultures as they can perform more complex metabolic tasks, seem to be more robust to environmental changes and produce different sets of metabolites of interest, which would normally be absent in mono-cultures.

The substrates currently used for production of microbial-derived teas mostly originate from raw carbons sources often specifically harvested from the environment for the sole purpose of producing plant and/or soil beneficial teas. Although they may be sustainably cultivated (such as kelp or alfalfa), they do not address (and may even exacerbate) the challenges associated with waste management. This invention described herein also addresses this challenge.

### BRIEF SUMMARY

In some aspects, provided herein are methods of preparing a high density culture of bacteria.

In some aspects, a method of preparing a high density culture of bacteria comprises the steps of (i) providing a bioreactor, wherein the bioreactor comprises a fermentation tank, a perfusion system, a medium, and a cell separator; (ii) preparing an inoculum, wherein the inoculum comprises a bacteria consortia, wherein the bacteria consortia comprises at least two bacteria species; (iii) inoculating the fermentation tank with the inoculum to form a fermentation culture; and (iv) growing the bacteria consortia until it has reached a target density. In some aspects, the spent medium in the fermentation tank is removed and fresh medium is added to the fermentation tank by the perfusion system while the bacteria consortia is growing. In some aspects, the target density is at least about 10¹² CFU/mL.

In some aspects, the method further comprises monitoring the fermentation culture while the bacteria consortia is growing. In some aspects, the fermentation culture is monitored for exponential growth of the bacteria consortia.

In some aspects, the monitoring is performed by a sensor.

In some aspects, the sensor is an Optical Density (OD) sensor, a pH probe, a dissolved oxygen probe, or a foam sensor.

In some aspects, the method further comprises harvesting the spent medium into a package.

In some aspects, the method further comprises harvesting the bacteria consortia into a package.

In some aspects, the method further comprises diluting the harvested content so that the density of the bacteria consortia in the package is about 10⁸ CFU/mL.

In some aspects, the harvesting occurs within about 7 days of inoculating the fermentation tank.

In some aspects, the method further comprises inoculating a second bioreactor with the harvested bacteria consortia.

In some aspects, provided herein is a method of preparing a high density culture of bacteria comprising the steps of (i) providing a bioreactor, wherein the bioreactor comprises a fermentation tank, a perfusion system, a medium, and a cell separator; (ii) preparing an inoculum, wherein the inoculum comprises a bacteria consortia, wherein the bacteria consortia comprises at least two aerobic bacteria species; (iii) inoculating the fermentation tank with the inoculum to form a fermentation culture; and (iv) growing the bacteria consortia until it has reached a target density, wherein spent medium in the fermentation tank is removed and fresh medium is added to the fermentation tank by the perfusion system while the bacteria consortia is growing, wherein the target density is at least about 10¹² CFU/mL.

In some aspects, the method further comprises harvesting the bacteria consortia.

In some aspects, the method further comprises combining the harvested bacteria consortia with an anaerobic bacteria consortia.

In some aspects, provided herein is a high density bacterial culture process that includes two or more of a) inoculating and growing individual fresh inoculum cultures comprising a target bacterial strain to late exponential phase; (to be grown to high density in a final fermentation vessel, where said inoculum cultures are seeded using freshly cultured isolates or subcultures of preserved stocks (cryopreserved, frozen, plated, refrigerated or otherwise dormant but viable stocks); b) separately combining a pre-processed organic slurry of plant material with a sufficient volume of sterile diluent to make a tea slurry; c) allowing nutrients to effuse out of the organic slurry into the liquid tea component of the tea slurry; d) harvesting the liquid tea from the tea slurry; e) eliminating plant-sourced microbes from the tea to make a sterile growth infusion; (using a micro-filtration, pasteurization or other sterilization methodology); f) setting up a sterile fermentation vessel with all required components to promote growth of the target bacteria; (comprising one or more of the following: buffers, anti-foaming agents, monitoring probes, recycling and aeration feeds, stirring mechanisms, and any appropriate growth components); g) adding sufficient media, sterile growth infusion, and inoculum cultures to the fermentation vessel making a fermentation culture; h) growing the fermentation culture to late exponential phase, where the fermentation culture comprises (i) an appropriate amount of culture medium designed for desired growth of the target bacterium in the fermentation vessel, (ii) a desired amount of nutrient rich growth infusion(s) (ratio(s) can be varied depending on desired results) at a pre-determined setup infusion-media ratio, and (iii) inoculate the fermentation vessel with a desired amount of fresh bacterial inoculum culture (Step a); i) initiating a media replacement regimen to replace spent media generated in the active fermentation culture with fresh media and sterile growth infusion, (whereby the spent culture medium of the fermentation culture is gradually and continuously harvested through a filtration system, leaving all target bacteria in the fermenter, while fresh growth infusion-media (which may have the same or a different ratio than the setup infusion-media ratio) is pumped into the fermentation vessel at a rate that may be the same as or different from the rate of spent culture medium harvesting); j) continuously harvesting the spent medium from the fermenter; k) monitoring the fermentation culture for exponential growth of the target bacteria; l) terminating the fermentation culture process when the target bacterial biomass is achieved; and m) harvesting the desired target bacterial cells from the fermenter. In some aspects, provided herein is a method of preparing a medium for the high density culture of bacteria comprising the steps of i) combining a pre-processed organic slurry of plant material with a sterile diluent to make a tea slurry; ii) allowing nutrients to effuse out of the pre-processed organic slurry into the liquid tea component of the tea slurry; iii) harvesting the liquid tea from the tea slurry; and iv) eliminating the plant-sourced microbes from the tea to make a sterile growth infusion, wherein a medium for the high density culture of bacteria comprises the sterile growth infusion. In some aspects, the tea slurry comprises a liquid tea component.

In some aspects, the plant sourced microbes are eliminated using micro-filtration or pasteurization.

In some aspects, provided herein is a high density culture of bacteria, produced by any of the methods described herein or any of the processes described herein.

In some aspects, the invention provided herein is a novel fermentation process for co-cultivation of microbes to produce beneficial secondary plant and/or soil products by recycling agriculture byproducts such as plant waste, bagasse or other byproducts of the beverage and food industry. Moreover, the process described herein reduces the scale-up footprint by over 1000-fold while cutting scale-up time by at least one third.

### BRIEF DESCRIPTION OF DRAWINGS

FIG. 1 (FIG. 1) shows a minimal footprint high density fermentation process design (MFHD). The fermentation tank (3) is less than 10 liters in volume. The liquid volume in the fermentation tank remains constant while fresh medium is fed through a peristaltic pump (2) from a medium tank (1), providing nutrients to the microbes as their density increases. Spent medium is continuously collected through a peristaltic pump (4), while bacteria cells are recycled into the fermentation tank via a hollow fiber module (5).

FIG. 2 (FIG. 2) shows a traditional volumetric fermentation scale-up. Fermentation volume increases 10-fold at each step (from 1 L at the beginning to 10000+ L) to achieve a final production scale fermentation. Smaller scale fermentation tanks are used to produce the next scale microbial inoculum via a seed train. CFU-Tᵢ represents the CFU/mL at inoculation, while CFU_Tₕ represents the CFU/mL at harvest.

FIG. 3 (FIG. 3) shows the MFHD process, in which an initial production inoculation of bacteria consortia of 10⁵ CFU/mL is grown to densities of 10¹²-10¹⁵ CFU/mL. The concentrated bacteria consortia and/or spent medium can be harvested directly and stored in small aliquots under temperature-controlled conditions for subsequent sale as product to customers or diluted to produce final product for sale. CFU-Tᵢ represents the CFU/mL at inoculation, while CFU_Tₕ represents the CFU/mL at harvest.

### DETAILED DESCRIPTION OF THE DISCLOSURE

Non-limiting examples of the various aspects are shown in the present disclosure.

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21- nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

The term “bioreactor” as used herein refers to a system containing a number of components directed to facilitating the culturing of bacteria. In some aspects, the bioreactor comprises a fermentation tank, a perfusion system, a medium, and a cell separator.

The terms “fermentation tank” or “fermenter” as used herein refer to a glass, plastic, or metal container that can provide a contained environment for culturing the microorganism. The vessel can be a standalone container or part of a larger system or setup. In some aspects, the fermentation tank is a one-time use fermentation tank. In some aspects, the fermentation tank is reusable.

The term “perfusion system” as used herein refers to a system that comprises one or more fluid handling mechanisms (e.g., peristaltic pumps) in order to facilitate and control the flow of media into the fermentation tank and out of the fermentation tank.

The terms “medium” or “media” as used herein are used in a broad sense, and refer to and encompasses a variety of solutions, buffers, formulations, and/or compounds, in which a microorganism or other type of biological samples or materials may reside for any period, of time that is conducive to the preservation of viability of the biological material placed within such buffers, solutions, formulations, and/or compounds.

The term “fresh medium” as used herein refers to medium that has not been previously provided to a culture of microorganisms and encompasses a variety of solutions, buffers, formulations, and/or compounds.

The term “spent medium” as used herein refers to medium that has been provided to a culture of microorganisms and has decreased nutrients as compared to fresh medium.

The term “cell separator” or “cell recycler” as used herein refers to an apparatus or membrane for separating bacteria cells from media. In some aspects, the cell separator separates the bacteria cells from spent media that is being removed from the bioreactor. In some aspects, the cell separator is a hollow fiber module.

The term “inoculation” as used herein refers to the addition of seeding cells to a medium to initiate culture. The term “inoculum” refers to the cells that are used to seed the medium to initiate culture.

The term “high density culture” as used herein refers to a culture of microorganisms that achieves densities between about 10¹² CFU/mL and about 10²⁰ CFU/mL. In some aspects, the culture achieves a density of between about 10¹² CFU/mL and about 10¹⁴ CFU/mL. In some aspects, the culture achieves a density of between about 10¹² CFU/mL and about 10¹³ CFU/mL. In some aspects, the culture achieves a density of between about 10¹³ CFU/mL and about 10¹⁵ CFU/mL. In some aspects, the culture achieves a density of between about 10¹⁴ CFU/mL and about 10¹⁵ CFU/mL. In some aspects, the culture achieves a density of between about 10¹² CFU/mL and about 10¹⁹ CFU/mL. In some aspects, the culture achieves a density of between about 10¹² CFU/mL and about 10¹⁸ CFU/mL. In some aspects, the culture achieves a density of between about 10¹² CFU/mL and about 10¹⁷ CFU/mL. In some aspects, the culture achieves a density of between about 10¹² CFU/mL and about 10¹⁶ CFU/mL. In some aspects, the culture achieves a density of between about 10¹² CFU/mL and about 10¹⁵ CFU/mL. In some aspects, the culture achieves a density of between about 10¹³ CFU/mL and about 10²⁰ CFU/mL. In some aspects, the culture achieves a density of between about 10¹⁴ CFU/mL and about 10²⁰ CFU/mL. In some aspects, the culture achieves a density of between about 10¹⁵ CFU/mL and about 10²⁰ CFU/mL. In some aspects, the culture achieves a density of between about 10¹⁶ CFU/mL and about 10²⁰ CFU/mL. In some aspects, the culture achieves a density of between about 10¹⁷ CFU/mL and about 10²⁰ CFU/mL. In some aspects, the culture achieves a density of between about 10¹⁸ CFU/mL and about 10²⁰ CFU/mL. In some aspects, the culture achieves a density of between about 10¹⁹ CFU/mL and about 10²⁰ CFU/mL. In some aspects, the culture achieves a density of about 10¹² CFU/mL. In some aspects, the culture achieves a density of about 10¹³ CFU/mL. In some aspects, the culture achieves a density of about 10¹⁴ CFU/mL. In some aspects, the culture achieves a density of about 10¹⁵ CFU/mL. In some aspects, the culture achieves a density of about 10¹⁶ CFU/mL. In some aspects, the culture achieves a density of about 10¹⁷ CFU/mL. In some aspects, the culture achieves a density of about 10¹⁸ CFU/mL. In some aspects, the culture achieves a density of about 10¹⁹ CFU/mL. In some aspects, the culture achieves a density of about 10²⁰ CFU/mL. In some aspects, the culture achieves a density greater than about 10¹² CFU/mL. In some aspects, the culture achieves a density greater than about 10¹³ CFU/mL. In some aspects, the culture achieves a density greater than about 10¹⁴ CFU/mL. In some aspects, the culture achieves a density greater than about 10¹⁵ CFU/mL. In some aspects, the culture achieves a density greater than about 10¹⁶ CFU/mL. In some aspects, the culture achieves a density greater than about 10¹⁷ CFU/mL. In some aspects, the culture achieves a density greater than about 10¹⁸ CFU/mL. In some aspects, the culture achieves a density greater than about 10¹⁹ CFU/mL.

The term “target density” as used herein refers to a density of microorganisms at which the microorganisms are harvested. In some aspects, the target density is at least 12 CFU/mL. In some aspects, the target density is about 10¹² CFU/mL. In some aspects, the target density is at least 10¹³ CFU/mL. In some aspects, the target density is about 10¹³ CFU/mL. In some aspects, the target density is at least 10¹⁴ CFU/mL. In some aspects, the target density is about 10¹⁴ CFU/mL. In some aspects, the target density is at least 10¹⁵ CFU/mL. In some aspects, the target density is about 10¹⁵ CFU/mL. In some aspects, the target density is at least 10¹¹ CFU/mL. In some aspects, the target density is about 10¹¹ CFU/mL. In some aspects, the target density is at least 10¹⁰ CFU/mL. In some aspects, the target density is about 10¹⁰ CFU/mL. In some aspects, the target density is at least 10⁹ CFU/mL. In some aspects, the target density is about 10⁹ CFU/mL. In some aspects, the target density is at least 10⁸ CFU/mL. In some aspects, the target density is about 10⁸ CFU/mL. In some aspects, the target density is at least 10⁷ CFU/mL. In some aspects, the target density is about 10⁷ CFU/mL. In some aspects, the target density is between about 10⁷ CFU/mL and about 10¹⁵ CFU/mL. In some aspects, the target density is between about 10⁸ CFU/mL and about 10¹⁵ CFU/mL. In some aspects, the target density is between about 10⁹ CFU/mL and about 10¹⁵ CFU/mL. In some aspects, the target density is between about 10¹⁰ CFU/mL and about 10¹⁵ CFU/mL. In some aspects, the target density is between about 10¹¹ CFU/mL and about 10¹⁵ CFU/mL. In some aspects, the target density is between about 10¹² CFU/mL and about 10¹⁵ CFU/mL. In some aspects, the target density is between about 10¹³ CFU/mL and about 10¹⁵ CFU/mL. In some aspects, the target density is between about 10¹⁴ CFU/mL and about 10¹⁵ CFU/mL. In some aspects, the target density is between about 10⁷ CFU/mL and about 10¹⁴ CFU/mL. In some aspects, the target density is between about 10⁷ CFU/mL and about 10¹³ CFU/mL. In some aspects, the target density is between about 10⁷ CFU/mL and about 10¹² CFU/mL. In some aspects, the target density is between about 10⁷ CFU/mL and about 10¹¹ CFU/mL. In some aspects, the target density is between about 10⁷ CFU/mL and about 10¹⁰ CFU/mL. In some aspects, the target density is between about 10⁷ CFU/mL and about 10⁹ CFU/mL. In some aspects, the target density is between about 10⁷ CFU/mL and about 10⁸ CFU/mL. In some aspects, the target density is between about 10¹³ CFU/mL and about 10¹⁵ CFU/mL. In some aspects, the target density is between about 10¹⁴ CFU/mL and about 10¹⁵ CFU/mL.

The term “sensor” as used herein refers to a probe that measures a variable of interest. In some aspects, the variable of interest is optical density of the fermentation culture. In some aspects, the variable of interest is pH level of the fermentation culture. In some aspects, the variable of interest is the dissolved oxygen concentration of the fermentation culture. In some aspects, the variable of interest is the foam levels of the fermentation culture.

The term “package” as used herein refers to any container suitable for the storage and/or transport of bacteria.

The terms “consortia” or “consortium” as used herein refer to at least two bacteria species. In some aspects, the bacteria consortia comprises two bacteria species. In some aspects, the bacteria consortia comprises three bacteria species. In some aspects, the bacteria consortia comprises four bacteria species. In some aspects, the bacteria consortia comprises five bacteria species. In some aspects, the bacteria consortia comprises six bacteria species. In some aspects, the bacteria consortia comprises seven bacteria species. In some aspects, the bacteria consortia comprises eight bacteria species. In some aspects, the bacteria consortia comprises nine bacteria species. In some aspects, the bacteria consortia comprises ten bacteria species. In some aspects, the bacteria consortia comprises eleven bacteria species. In some aspects, the bacteria consortia comprises twelve bacteria species. In some aspects, the bacteria consortia comprises thirteen bacteria species. In some aspects, the bacteria consortia comprises fourteen bacteria species. In some aspects, the bacteria consortia comprises fifteen bacteria species. In some aspects, the bacteria consortia comprises sixteen bacteria species. In some aspects, the bacteria consortia comprises seventeen bacteria species. In some aspects, the bacteria consortia comprises eighteen bacteria species. In some aspects, the bacteria consortia comprises nineteen bacteria species. In some aspects, the bacteria consortia comprises twenty bacteria species. In some aspects, the bacteria consortia comprises twenty-one bacteria species. In some aspects, the bacteria consortia comprises twenty-two bacteria species. In some aspects, the bacteria consortia comprises twenty-three bacteria species. In some aspects, the bacteria consortia comprises twenty-four bacteria species. In some aspects, the bacteria consortia comprises twenty-five bacteria species.

The term “tea” as used herein refers to the bacteria-free product of fermenting plant and/or compost infusions. The tea contains metabolites secreted by bacteria during fermentation as well as fermentation byproducts beneficial to plants and/or soil microbiomes. In some aspects, the metabolite is a phytohormone. In some aspects, the phytohormone is Indole-3-acetic acid. In some aspects, the phytohormone is an Indole-3-acetic acid analogue. In some aspects, the metabolite is an organic acid. In some aspects, the organic acid is an amino acid. In some aspects, the organic acid is a lactic acid. The term “slurry” as used herein refers to the mixture of plant and/or compost material and water.

### II. Method of Preparing a High Density Culture of Bacteria

The present disclosure provides methods of preparing a high density culture of bacteria.

In some aspects, a method of preparing a high density culture of bacteria comprises the steps of (i) providing a bioreactor, wherein the bioreactor comprises a fermentation tank, a perfusion system, a medium, and a cell separator; (ii) preparing an inoculum, wherein the inoculum comprises a bacteria consortia, wherein the bacteria consortia comprises at least two bacteria species; (iii) inoculating the fermentation tank with the inoculum to form a fermentation culture; and (iv) growing the bacteria consortia until it has reached a target density. In some aspects, the spent medium in the fermentation tank is removed and fresh medium is added to the fermentation tank by the perfusion system while the bacteria consortia is growing.

In some aspects, a method of preparing a high density culture of bacteria comprises the steps of (i) providing a bioreactor, wherein the bioreactor comprises a fermentation tank, a perfusion system, a medium, and a cell separator; (ii) preparing an inoculum, wherein the inoculum comprises a single bacteria species; (iii) inoculating the fermentation tank with the inoculum to form a fermentation culture; and (iv) growing the bacteria consortia until it has reached a target density. In some aspects, the spent medium in the fermentation tank is removed and fresh medium is added to the fermentation tank by the perfusion system while the bacteria consortia is growing.

In some aspects, the target density is at least about 10¹² CFU/mL. In some aspects, the target density is about 10¹² CFU/mL. In some aspects, the target density is at least about 10¹³ CFU/mL. In some aspects, the target density is about 10¹³ CFU/mL. In some aspects, the target density is at least about 10¹⁴ CFU/mL. In some aspects, the target density is about 10¹⁴ CFU/mL. In some aspects, the target density is at least about 10¹⁵ CFU/mL. In some aspects, the target density is about 10¹⁵ CFU/mL. In some aspects, the target density is between about 10¹² CFU/mL and about 10¹⁵ CFU/mL. In some aspects, the target density is between about 10¹³ CFU/mL and about 10¹⁵ CFU/mL. In some aspects, the target density is between about 10¹⁴ CFU/mL and about 10¹⁵ CFU/mL. In some aspects, the target density is between about 10¹² CFU/mL and about 10¹⁴ CFU/mL. In some aspects, the target density is between about 10¹² CFU/mL and about 10¹³ CFU/mL.

In some aspects, the bacteria consortia comprises at least one bacteria species selected from the group consisting of Clostridium pasteurianum, Clostridium beijerinckii, Lactobacillus acidophilus, and Lactobacillus plantarum.

In some aspects, the bacteria consortia comprises at least one bacteria species selected from the group consisting of Azotobacter vinelandii, Bacillus megaterium, Bacillus licheniformis, and Bacillus amyloliquefaciens.

In some aspects, the bacteria consortia comprises at least one bacteria from the genera Clostridium, Lactobacillus, or Enterobacter.

In some aspects, the bacteria consortia comprises at least one bacteria from the genera Bacillus, Pseudomonas, Streptomyces, or Azotobacter.

In some aspects, the bacteria consortia comprises at least one bacteria from the genera Bacillus, Azospirillum, Pseudomonas, Lactobacillus, Desulfococcus, Desulfotomaculum, Marinobacter, Nitrosopumilus, Ruminococcus, Aquabacterium, Leptolyngbya, Leptospirillum, Paenibacillus, Microcoleus, Clostridium, Xenococcus, Acetobacter, Candidatus, Methanosaeta, and Brevibacillus.

In some aspects, the bacteria consortia comprises at least one bacteria from the genera Azospirillum, Pseudomonas, Lactobacillus, Marinobacter, Nitrosopumilus, Aquabacterium, Leptolyngbya, Leptospirillum, Microcoleus, Xenococcus, Acetobacter, Brevibacillus and Paenibacillus.

In some aspects, the bacteria consortia comprises at least one bacteria from the genera Desulfococcus, Desulfotomaculum, Ruminococcus, Clostridium, and Methanosaeta and Candidatus.

In some aspects, the bacteria consortia comprises at least one bacteria that is an obligate aerobe. In some aspects, the bacteria consortia comprises at least one bacteria that is a facultative anaerobe. In some aspects, the bacteria consortia comprises at least one bacteria that is an obligate anaerobe.

In some aspects, the bacteria consortia comprises at least one bacteria from the genera Xanthobacter, Azorhizobium, Aquabacter, Methylobacterium, Ralstonia, and Starkeya.

In some aspects, the bacteria described herein have beneficial traits as shown in

TABLE 1

| Bacteria | Traits | Reference |
|---|---|---|
| Clostridium | N2 fixation | KASAP, M., & CHEN, J-S., “Clostridium pasteurianum W5 |
| Pasteurianum | | synthesizes two NifH-related polypeptides under nitrogen-fixing |
| | | conditions,” Microbiology 151:2353-2362, Microbiology Society, |
| | | United Kingdom (July 2005). |
| | | CHEN, J-S., “Nitrogen Fixation in the Clostridia,” in Genetics and |
| | | Regulation of Nitrogen Fixation in Free-Living Bacteria, Klipp, W., |
| | | et al., eds., pp. 53-64, Springer, United States (2005). |
| Clostridium | N2 fixation | ROSENBLUM, E. D., & WILSON, P. W., “Fixation Of Isotopic |
| Beijerinckii | | Nitrogen By Clostridium,” Journal of Bacteriology 57(4):413-414, |
| | | American Society for Microbiology, United States (1949). |
| | | CHEN, J-S., “Nitrogen Fixation in the Clostridia,” in Genetics and |
| | | Regulation of Nitrogen Fixation in Free-Living Bacteria, Klipp, W., |
| | | et al., eds., pp. 53-64, Springer, United States (2005). |
| Lactobacillus | Plant | HUSAIN, A., et al., “Antifungal Activity of Lactic Acid Bacteria |
| acidophilus | protection | Isolated from Soil Rhizosphere on Fusarium Species Infected Chilli |
| | | Seeds,” American Scientific Research Journal for Engineering, |
| | | Technology, and Sciences 29(1): 182-202, Global Society of |
| | | Scientific Research and Researchers, Jordan (2017). |
| Lactobacillus | Plant | HUSAIN, A., et al., “Antifungal Activity of Lactic Acid Bacteria |
| plantarum | protection | Isolated from Soil Rhizosphere on Fusarium Species Infected Chilli |
| | | Seeds,” American Scientific Research Journal for Engineering, |
| | | Technology, and Sciences 29(1): 182-202, Global Society of Scientific |
| | | Research and Researchers, Jordan (2017). |
| Azotobacter | N2 fixation | BISHOP, P. E., et al., “Expression of an alternative nitrogen fixation |
| vinelandii | | system in Azotobacter vinelandii,” Journal of Bacteriology |
| | | 150(3):1244-1251, American Society of Microbiology, United |
| | | States (1982). |
| | | NOAR, J. D., & BRUNO-BARCENA, J. M., “Azotobacter |
| | | vinelandii: the source of 100 years of discoveries and many more to |
| | | come,” Microbiology 164(4):421-436, Microbiology Society, United |
| | | Kingdom (2018). |
| Bacillus | Plant | LOPEZ-BUCIO, J., et al., “Bacillus megaterium Rhizobacteria |
| megaterium | growth | Promote Growth and Alter Root-System Architecture Through an |
| | promoting | Auxin- and Ethylene-Independent Signaling Mechanism in |
| | | Arabidopsis thaliana,” Mol Plant Microbe Interact 20(2):207-217, |
| | | The American Phytopathological Society, United States (2007). |
| Bacillus | Plant | SUKKASEM, P., et al., “A multifaceted rhizobacterium Bacillus |
| licheniformis | growth | licheniformis functions as a fungal antagonist and a promoter of |
| | promoting | plant growth and abiotic stress tolerance,” Environmental and |
| | | Experimental Botany 155:541-551, Elsevier, Netherlands (2018). |
| Bacillus | IAA | MOHITE, B., “Isolation and characterization of indole acetic acid |
| amyloliquefaciens | production | (IAA) producing bacteria from rhizospheric soil and its effect on |
| | | plant growth,” Journal of Soil Science and Plant Nutrition |
| | | 13(3):638-649, Sociedad Chilena de la Ciencia del Suelo, Chile |
| | | (2013). |

In some aspects, the method further comprises monitoring the fermentation culture while the bacteria consortia is growing. In some aspects, the fermentation culture is monitored for exponential growth of the bacteria consortia.

In some aspects, the monitoring is performed by a sensor.

In some aspects, the sensor is an Optical Density (OD) sensor, a pH probe, a dissolved oxygen probe, or a foam sensor.

In some aspects, the method further comprises harvesting the spent medium into a package. In some aspects, the method further comprises harvesting the bacteria consortia into a package. In some aspects, the bacteria consortia is killed prior to harvesting the spent medium. In some aspects, the bacteria consortia is killed by temperature, osmotic, or chemical means.

In some aspects, the method further comprises diluting the harvested content so that the density of the bacteria consortia in the package is about 10⁷ CFU/mL. In some aspects, the method further comprises diluting the harvested content so that the density of the bacteria consortia in the package is at least about 10⁷ CFU/mL. In some aspects, the method further comprises diluting the harvested content so that the density of the bacteria consortia in the package is about 10⁸ CFU/mL. In some aspects, the method further comprises diluting the harvested content so that the density of the bacteria consortia in the package is at least about 10⁸ CFU/mL. In some aspects, the method further comprises diluting the harvested content so that the density of the bacteria consortia in the package is about 10⁹ CFU/mL. In some aspects, the method further comprises diluting the harvested content so that the density of the bacteria consortia in the package is at least about 10⁹ CFU/mL. In some aspects, the method further comprises diluting the harvested content so that the density of the bacteria consortia in the package is between about 10⁷ CFU/mL and about 10⁹ CFU/mL. In some aspects, the method further comprises diluting the harvested content so that the density of the bacteria consortia in the package is between about 10⁸ CFU/mL and about 10⁹ CFU/mL. In some aspects, the method further comprises diluting the harvested content so that the density of the bacteria consortia in the package is between about 10⁷ CFU/mL and about 10⁸ CFU/mL.

In some aspects, the harvesting occurs within about 7 days of inoculating the fermentation tank. In some aspects, the harvesting occurs within about 4, 5, 6, 7, 8, 9, 10, 11, 12, 13, 14, 15, 16, 17, 18, 19, 20, 21, 22, 23, 24, 25, 26, 27, 28, 29 or 30 days of inoculating the fermentation tank. In some aspects, the harvesting occurs between about 4 days and about 10 days of inoculating the fermentation tank. In some aspects, the harvesting occurs between about 4 days and about 7 days of inoculating the fermentation tank. In some aspects, the harvesting occurs between about 5 days and about 7 days of inoculating the fermentation tank. In some aspects, the harvesting occurs between about 6 days and about 7 days of inoculating the fermentation tank. In some aspects, the harvesting occurs between about 7 days and about 8 days of inoculating the fermentation tank. In some aspects, the harvesting occurs between about 7 days and about 9 days of inoculating the fermentation tank. In some aspects, the harvesting occurs between about 7 days and about 10 days of inoculating the fermentation tank.

In some aspects, the harvesting occurs between about 4 days and about 30 days of inoculating the fermentation tank. In some aspects, the harvesting occurs between about 4 days and about 25 days of inoculating the fermentation tank. In some aspects, the harvesting occurs between about 4 days and about 20 days of inoculating the fermentation tank. In some aspects, the harvesting occurs between about 4 days and about 15 days of inoculating the fermentation tank. In some aspects, the harvesting occurs between about 4 days and about 14 days of inoculating the fermentation tank. In some aspects, the harvesting occurs between about 4 days and about 13 days of inoculating the fermentation tank. In some aspects, the harvesting occurs between about 4 days and about 12 days of inoculating the fermentation tank. In some aspects, the harvesting occurs between about 4 days and about 11 days of inoculating the fermentation tank. In some aspects, the harvesting occurs between about 5 days and about 30 days of inoculating the fermentation tank. In some aspects, the harvesting occurs between about 6 days and about 30 days of inoculating the fermentation tank. In some aspects, the harvesting occurs between about 7 days and about 30 days of inoculating the fermentation tank. In some aspects, the harvesting occurs between about 8 days and about 30 days of inoculating the fermentation tank. In some aspects, the harvesting occurs between about 9 days and about 30 days of inoculating the fermentation tank. In some aspects, the harvesting occurs between about 10 days and about 30 days of inoculating the fermentation tank. In some aspects, the harvesting occurs between about 11 days and about 30 days of inoculating the fermentation tank. In some aspects, the harvesting occurs between about 12 days and about 30 days of inoculating the fermentation tank. In some aspects, the harvesting occurs between about 13 days and about 30 days of inoculating the fermentation tank. In some aspects, the harvesting occurs between about 14 days and about 30 days of inoculating the fermentation tank. In some aspects, the harvesting occurs between about 15 days and about 30 days of inoculating the fermentation tank. In some aspects, the harvesting occurs between about 20 days and about 30 days of inoculating the fermentation tank. In some aspects, the harvesting occurs between about 25 days and about 30 days of inoculating the fermentation tank.

In some aspects, the harvested bacteria is stored in liquid form at room temperature. In some aspects, the harvested bacteria is stored at about 4° C. In some aspects, the harvested bacteria is frozen. In some aspects, the harvested bacteria is dried.

In some aspects, the method further comprises inoculating a second bioreactor with the harvested bacteria consortia.

In some aspects, provided herein is a method of preparing a high density culture of bacteria comprising the steps of (i) providing a bioreactor, wherein the bioreactor comprises a fermentation tank, a perfusion system, a medium, and a cell separator; (ii) preparing an inoculum, wherein the inoculum comprises a bacteria consortia, wherein the bacteria consortia comprises at least two aerobic bacteria species; (iii) inoculating the fermentation tank with the inoculum to form a fermentation culture; and (iv) growing the bacteria consortia until it has reached a target density, wherein spent medium in the fermentation tank is removed and fresh medium is added to the fermentation tank by the perfusion system while the bacteria consortia is growing, wherein the target density is at least about 10¹² CFU/mL.

In some aspects, the method further comprises harvesting the bacteria consortia. In some aspects, the at least two aerobic bacteria species are selected from the group consisting of Azotobacter vinelandii, Bacillus megaterium, Bacillus licheniformis, and Bacillus amyloliquefaciens. In some aspects, the at least two aerobic bacteria species comprises at least one bacteria from the genera Bacillus, Pseudomonas, Streptomyces, or Azotobacter.

In some aspects, the method further comprises combining the harvested bacteria consortia with an anaerobic bacteria consortia.

In some aspects, the anaerobic bacteria consortia comprises at least one bacteria species selected from the group consisting of Clostridium pasteurianum, Clostridium beijerinckii, Lactobacillus acidophilus, and Lactobacillus plantarum.

In some aspects, the anaerobic bacteria consortia comprises at least one bacteria from the genera Clostridium, Lactobacillus, or Enterobacter.

In some aspects, provided herein is a high density bacterial culture process that includes two or more of a) inoculating and growing individual fresh inoculum cultures comprising a target bacterial strain to late exponential phase; (to be grown to high density in a final fermentation vessel, where said inoculum cultures are seeded using freshly cultured isolates or subcultures of preserved stocks (cryopreserved, frozen, plated, refrigerated or otherwise dormant but viable stocks); b) separately combining a pre-processed organic slurry of plant material with a sufficient volume of sterile diluent to make a tea slurry; c) allowing nutrients to effuse out of the organic slurry into the liquid tea component of the tea slurry; d) harvesting the liquid tea from the tea slurry; e) eliminating plant-sourced microbes from the tea to make a sterile growth infusion; (using a micro-filtration, pasteurization or other sterilization methodology); f) setting up a sterile fermentation vessel with all required components to promote growth of the target bacteria; (comprising one or more of the following: buffers, anti-foaming agents, monitoring probes, recycling and aeration feeds, stirring mechanisms, and any appropriate growth components); g) adding sufficient media, sterile growth infusion, and inoculum cultures to the fermentation vessel making a fermentation culture; h) growing the fermentation culture to late exponential phase, where the fermentation culture comprises (i) an appropriate amount of culture medium designed for desired growth of the target bacterium in the fermentation vessel, (ii) a desired amount of nutrient rich growth infusion(s) (ratio(s) can be varied depending on desired results) at a pre-determined setup infusion-media ratio, and (iii) inoculate the fermentation vessel with a desired amount of fresh bacterial inoculum culture (Step a); i) initiating a media replacement regimen to replace spent media generated in the active fermentation culture with fresh media and sterile growth infusion, (whereby the spent culture medium of the fermentation culture is gradually and continuously harvested through a filtration system, leaving all target bacteria in the fermenter, while fresh growth infusion-media (which may have the same or a different ratio than the setup infusion-media ratio) is pumped into the fermentation vessel at a rate that may be the same as or different from the rate of spent culture medium harvesting); j) continuously harvesting the spent medium from the fermenter; k) monitoring the fermentation culture for exponential growth of the target bacteria; l) terminating the fermentation culture process when the target bacterial biomass is achieved; and m) harvesting the desired target bacterial cells from the fermenter.

In some aspects, provided herein is a method of preparing a medium for the high density culture of bacteria comprising the steps of i) combining a pre-processed organic slurry of plant material with a sterile diluent to make a tea slurry; ii) allowing nutrients to effuse out of the pre-processed organic slurry into the liquid tea component of the tea slurry; iii) harvesting the liquid tea from the tea slurry; and iv) eliminating the plant-sourced microbes from the tea to make a sterile growth infusion, wherein a medium for the high density culture of bacteria comprises the sterile growth infusion. In some aspects, the tea slurry comprises a liquid tea component.

In some aspects, the plant sourced microbes are eliminated using micro-filtration or pasteurization.

In some aspects, provided herein is a high density culture of bacteria, produced by any of the methods described herein or any of the processes described herein.

In some aspects, the minimum-scale high density fermentation process provided herein comprises at least five steps.

### Step 1a: Inoculum Preparation

In some aspects, the desired axenic microbial strains derived from the cryopreserved working cell bank are revived on semi-solid agar medium using the plate streak method. In some aspects, an isolated colony for each strain is subsequently amplified in <50 mL of sterile liquid medium under appropriate growth conditions. In some aspects, the microbial strains are derived from a cell bank. In some aspects, the microbial strains are cryopreserved. In some aspects, the microbial strains are revived on an agar plate. In some aspects, a colony is isolated from the agar plate. In some aspects, the isolated colony is amplified in a sterile liquid medium. In some aspects, the isolated colony is amplified in about 50 mL of a sterile liquid medium. In some aspects, the isolated colony is amplified in about 25 mL of a sterile liquid medium. In some aspects, the isolated colony is amplified in about 10 mL of a sterile liquid medium. In some aspects, the isolated colony is amplified in about 5 mL of a sterile liquid medium. In some aspects, the isolated colony is amplified in about 1 mL of a sterile liquid medium.

### Step 1b: Medium Preparation

In some aspects, the fermentation medium is produced by autoclaving, pasteurizing microfiltration or steeping byproducts. In some aspects, the byproducts are agriculture byproducts. In some aspects, the agriculture byproducts include plant waste and/or composts. In some aspects, the byproducts are from the beverage/food industry. In some aspects, autoclaving, pasteurizing microfiltration or steeping byproducts produces an infusion rich in nutrients to support microbial growth. In some aspects, the infusions are subsequently sterilized by filtration or pasteurization or other methods that deactivate or eliminate endogenous microbes/viruses. In some aspects, the infusion to be fermented is derived from either one source or multiple sources combined at different ratios to produce the fermentation medium.

### Step 2: Inoculation of the Fermentation Tank

In some aspects, Step 2 concerns inoculation of the fermentation tank. In some aspects, a clean benchtop fermentation tank is filled with appropriate production medium and assembled with agitator and all desired probes. In some aspects, the clean benchtop fermentation tank has a volume of about 1 L, 2 L, 3 L, 4 L, or 5 L. In some aspects, the probes are selected from the group consisting of an OD sensor, a pH probe, a dissolved oxygen probe and a foam sensor. In some aspects, the assembled fermentation tank is sterilized by autoclaving. In some aspects, the assembled fermentation tank is connected to a controller module. In some aspects, the gas, medium and other reagents (acid, base, antifoam) lines are then hooked-up. The fermentation tank type, e.g. one-time use fermentation tank or classic re-usable fermentation tank, is selected based on the final product/customer requirements. Based on the microbial consortium to be produced, the fermentation conditions are set in advance of the inoculation, e.g. pH, temperature, agitation, gas flow rate. In some aspects, antifoam is added and the cell recycle system is turned on to equilibrate all components of the fermentation tank with the desired microbial growth conditions. In some aspects, each strain is subsequently inoculated and allowed to grow until reaching the desired concentration or target density in the fermentation tank. In some aspects, the desired concentration is determined based on the strain's growth profile and requirements for co-cultivation in a given consortium. In some aspects, the requirements for co-cultivation is determined experimentally prior to establishing the manufacturing process. In some aspects, multiple inoculations are performed, e.g. a given microbe or group of microbes is added at different times during the span of the fermentation process.

### Step 3: Fermentation

In some aspects, once the co-cultivated microbes reach the limit of the exponential growth phase and prior to the stationary phase, the medium permeate stream (Mₚₑᵣₘ) is initiated together with addition of fresh sterile medium (M_{fresh}). In some aspects, the cell retention time (CRT) in the vessel is as long as needed until harvest, while medium retention time (MRT), is adjusted to control cell density. In some aspects, the MRT is decreased, which leads to a corresponding increase in cell density. In some aspects, the CRT decreases as needed to maintain the desired microbial population equilibrium. In some aspects the CRT is decreased by maintaining the M_{fresh} flow rate (M_{fresh}FR) constant while decrease the Mₚₑᵣₘ flow rate (MₚₑᵣₘFR).

### Step 4a, Harvest of the Microbes

In some aspects, once the desired high cell density is achieved the fermentation is terminated. In some aspects, the content of the fermentation tank is dispensed aseptically into appropriately sized bottles and packaged. In some aspects, prior to bottling the microbes are deactivated or killed by temperature, osmotic or chemical means.

### Step 4b, Harvest of the Fermented Medium

In some aspects, within than 7 days, the resulting tea, i.e. microbe free-fermentate or Mₚₑᵣₘ, is continuously harvested as part of the fermentation process. In some aspects, the resulting tea is continuously harvested until the fermentation process is terminated. In some aspects, the resulting tea is continuously harvested within 3, 4, 5, 6, 7, 8, 9, 10, 11, 12, 13, 14, 15, 16, 17, 18, 19, 20, 21, 22, 23, 24, 25, 26, 27, 28, or 29 days of inoculation. In some aspects, the resulting tea is continuously harvested between about 3 days and about 9 days of inoculation. In some aspects, the resulting tea is continuously harvested between about 4 days and about 9 days of inoculation. In some aspects, the resulting tea is continuously harvested between about 5 days and about 9 days of inoculation. In some aspects, the resulting tea is continuously harvested between about 6 days and about 9 days of inoculation. In some aspects, the resulting tea is continuously harvested between about 7 days and about 9 days of inoculation. In some aspects, the resulting tea is continuously harvested between about 8 days and about 9 days of inoculation. In some aspects, the resulting tea is continuously harvested between about 3 days and about 8 days of inoculation. In some aspects, the resulting tea is continuously harvested between about 3 days and about 7 days of inoculation. In some aspects, the resulting tea is continuously harvested between about 3 days and about 6 days of inoculation. In some aspects, the resulting tea is continuously harvested between about 3 days and about 5 days of inoculation. In some aspects, the resulting tea is continuously harvested between about 3 days and about 4 days of inoculation.

In some aspects, the resulting tea is continuously harvested between about 3 days and about 29 days of inoculation. In some aspects, the resulting tea is continuously harvested between about 3 days and about 27 days of inoculation. In some aspects, the resulting tea is continuously harvested between about 3 days and about 24 days of inoculation. In some aspects, the resulting tea is continuously harvested between about 3 days and about 21 days of inoculation. In some aspects, the resulting tea is continuously harvested between about 3 days and about 18 days of inoculation. In some aspects, the resulting tea is continuously harvested between about 3 days and about 15 days of inoculation. In some aspects, the resulting tea is continuously harvested between about 3 days and about 14 days of inoculation. In some aspects, the resulting tea is continuously harvested between about 3 days and about 13 days of inoculation. In some aspects, the resulting tea is continuously harvested between about 3 days and about 12 days of inoculation. In some aspects, the resulting tea is continuously harvested between about 3 days and about 11 days of inoculation. In some aspects, the resulting tea is continuously harvested between about 3 days and about 10 days of inoculation. In some aspects, the resulting tea is continuously harvested between about 4 days and about 29 days of inoculation. In some aspects, the resulting tea is continuously harvested between about 5 days and about 29 days of inoculation. In some aspects, the resulting tea is continuously harvested between about 6 days and about 29 days of inoculation. In some aspects, the resulting tea is continuously harvested between about 7 days and about 29 days of inoculation. In some aspects, the resulting tea is continuously harvested between about 8 days and about 29 days of inoculation. In some aspects, the resulting tea is continuously harvested between about 9 days and about 29 days of inoculation. In some aspects, the resulting tea is continuously harvested between about 10 days and about 29 days of inoculation. In some aspects, the resulting tea is continuously harvested between about 11 days and about 29 days of inoculation. In some aspects, the resulting tea is continuously harvested between about 12 days and about 29 days of inoculation. In some aspects, the resulting tea is continuously harvested between about 13 days and about 29 days of inoculation. In some aspects, the resulting tea is continuously harvested between about 14 days and about 29 days of inoculation. In some aspects, the resulting tea is continuously harvested between about 15 days and about 29 days of inoculation. In some aspects, the resulting tea is continuously harvested between about 18 days and about 29 days of inoculation. In some aspects, the resulting tea is continuously harvested between about 21 days and about 24 days of inoculation. In some aspects, the resulting tea is continuously harvested between about 27 days and about 29 days of inoculation.

In some aspects, priority is given to the Mₚₑᵣₘ that is derived from the fermentation time point where the cell density is at its highest levels. In some aspects, priority is given to the Mₚₑᵣₘ that is derived from the fermentation time point when the microbial population is at the desired equilibrium. In some aspects, the microbes are separated from the tea after fermentation shutdown. In some aspects, the microbes are separated though a filtration or centrifugation process.

In some aspects, the products of Steps 4a and 4b are stored in liquid form at room temperature, about 4° C. or frozen depending on the consortium and end use. In some aspects, the products are dried prior to marketing.

### Step 5 Packaging

In some aspects, the product derived from the fermentation process described in Steps 1-4 is subsequently diluted using inert diluents. In some aspects, the inert diluents are for liquid forms of the fermentate. In some aspects, the inert diluent for the liquid form of the fermentate is water. In some aspects, the inert diluents are for solid forms of the fermentate. In some aspects, the inert diluent for the solid form of the fermentate is a cellulosic powder. In some aspects, the diluents are selected for their ability to maintain the microbial population in the stationary phase, limiting alterations of the strains' ratio in the final product. In some aspects, the dilution factor is at least 1:10,000. In some aspects, the dilution factor is about 1:10,000. In some aspects, the dilution produces a microbial consortium with an average density of about 1×10⁸ CFU/mL. In some aspects, the dilution produces a microbial consortium with a density of about 1×10⁸ CFU/mL. In some aspects, the dilution produces a microbial consortium with a density of at least about 1×10⁸ CFU/mL. In some aspects, the dilution produces a microbial consortium with an average density of about 1×10⁹ CFU/mL. In some aspects, the dilution produces a microbial consortium with a density of about 1×10⁹ CFU/mL. In some aspects, the dilution produces a microbial consortium with a density of at least about 1×10⁹ CFU/mL. In some aspects, the dilution produces a microbial consortium with an average density of about 1×10⁷ CFU/mL. In some aspects, the dilution produces a microbial consortium with a density of about 1×10⁷ CFU/mL. In some aspects, the dilution produces a microbial consortium with a density of at least 1×10⁷ CFU/mL.

In some aspects, the dilution produces a microbial consortium with an average density between about 1×10⁷ CFU/mL and about 1×10⁹ CFU/mL. In some aspects, the dilution produces a microbial consortium with an average density between about 1×10⁷ CFU/mL and about 1×10⁸ CFU/mL. In some aspects, the dilution produces a microbial consortium with an average density between about 1×10⁸ CFU/mL and about 1×10⁹ CFU/mL.

In some aspects, the dilution factor is adjusted based on the final concentration of the microbes at the end of fermentation. In some aspects, the dilution factor is adjusted to achieve the desired final levels of microbes.

In some aspects, the dilutions factors range from about 1:10,000 to about 1:1,000,000. In some aspects, the dilutions factors range from about 1:10,000 to about 1:1,000,000. In some aspects, the dilutions factors range from about 1:25,000 to about 1:1,000,000. In some aspects, the dilutions factors range from about 1:50,000 to about 1:1,000,000. In some aspects, the dilutions factors range from about 1:75,000 to about 1:1,000,000. In some aspects, the dilutions factors range from about 1:100,000 to about 1:1,000,000. In some aspects, the dilutions factors range from about 1:200,000 to about 1:1,000,000. In some aspects, the dilutions factors range from about 1:300,000 to about 1:1,000,000. In some aspects, the dilutions factors range from about 1:400,000 to about 1:1,000,000. In some aspects, the dilutions factors range from about 1:500,000 to about 1:1,000,000. In some aspects, the dilutions factors range from about 1:600,000 to about 1:1,000,000. In some aspects, the dilutions factors range from about 1:700,000 to about 1:1,000,000. In some aspects, the dilutions factors range from about 1:800,000 to about 1:1,000,000. In some aspects, the dilutions factors range from about 1:900,000 to about 1:1,000,000. In some aspects, the dilutions factors range from about 1:10,000 to about 1:900,000. In some aspects, the dilutions factors range from about 1:10,000 to about 1:800,000. In some aspects, the dilutions factors range from about 1:10,000 to about 1:700,000. In some aspects, the dilutions factors range from about 1:10,000 to about 1:600,000. In some aspects, the dilutions factors range from about 1:10,000 to about 1:500,000. In some aspects, the dilutions factors range from about 1:10,000 to about 1:400,000. In some aspects, the dilutions factors range from about 1:10,000 to about 1:300,000. In some aspects, the dilutions factors range from about 1:10,000 to about 1:200,000. In some aspects, the dilutions factors range from about 1:10,000 to about 1:100,000. In some aspects, the dilutions factors range from about 1:10,000 to about 1:75,000. In some aspects, the dilutions factors range from about 1:10,000 to about 1:50,000. In some aspects, the dilutions factors range from about 1:10,000 to about 1:25,000.

Thus, within a week, a 1 L fermentation tank using the process described herein produces the same amount of final product than a 10,000 L-1,000,000 L fermentation tank using other processes. In some aspects, diluted products are packaged in differently sized containers. In some aspects, the packages are based on market demand.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

Any examples provided herein are offered by way of illustration and not by way of limitation.

### EXAMPLES

### Example 1

### High Density Aerobic Fermentation of Infusions Using a Microbial Consortium of Four Isolates

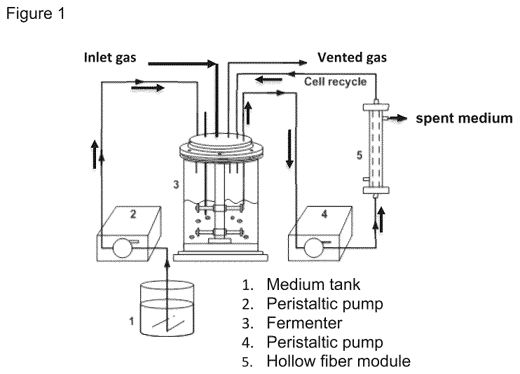

Infusions are derived from agriculture byproducts such as plant waste and/or composts or from the byproducts of the beverage/food industry. A plant-derived infusion is produced by steeping 25-50 g of dried hemp or hops leaves in 1 L of 60°C.-80 ° C. water for 1 hour. Plant material is placed in a cheese cloth prior to steeping for easy removal. After steeping, 1.5 L of the suspension is transferred to a 2 L glass fermenter vessel (Bioflo 120, Eppendorf USA) and autoclaved at 121° C. for 30 min. Compost infusions are processed in a similar manner. Once cooled to 25°C.-35° C., individually grown saturated axenic cultures of Bacillus licheniformis, Bacillus megaterium, and Bacillus amyloliquefaciens are used to inoculate the fermenter to achieve a concentration for each stain of 10⁵ colony forming units (CFUs) per mL. Air flow and agitation are automatically adjusted to maintain a dissolved oxygen level of 25-30% during the length of the fermentation 3-7 days. pH is maintained between 7-9 using a 20% phosphoric acid and 0.1 N KOH, as acid and base solutions respectively. Cells in the fermenter are maintained in logarithmic growth phase through continuous addition of fresh medium to the fermenter. A cell recycle system comprised of a 0.2 micron hollow fiber is used to remove the spent medium while maintaining the cells in the fermenter. Medium retention time varies over the length of the fermentation run ranging from a steady state at the start of the process to ≤12 hours at the end of the run. FIG. 1 (FIG. 1) shows an exemplary minimal footprint high density fermentation process design (MFHD).

At the conclusion of the fermentation, total CFUs are determined by plating an aliquot for the fermentate on nutrient agar plates and colonies are counted. Estimated total CFUs are >10¹² per mL. Alternatively, the number of cells per bacteria strain is determined using digital PCR (Bio-Rad, USA) with bacteria isolate specific primers (G. Gobert et al., J. Microbiol. Methods Vol. 128, 2018; M. Ricchi et al., Front. Microbiol. Vol. 28, 2017).

### Example 2

### High density Anaerobic Fermentation of a Plant-Derived Infusion Using a Microbial Consortium of Four (4) Isolates

A plant and/or compost-derived infusion is prepared as described in Example 1. Anaerobic or facultative anaerobic bacteria isolates such as but not limited to Clostridium beijerinckii, Clostridium pasteurianum, lactobacillus plantarum, lactobacillus acidophilus are grown under anaerobic conditions. These microbes are selected as they are known to have plant and/or soil beneficial traits as described in Table 1. In lieu of air, nitrogen gas is used during fermentation acting as a source of nitrogen for diazotroph bacteria such as C. beijerinckii and C. pasteurianum and an oxygen displacement gas. Dissolved oxygen is maintained <5% for the span of the fermentation (3-7 days) while gas flow and agitation rates are controlled based on conductivity and/or cell density monitored using an in-line optical density probe and/or regular sampling using a benchtop spectrophotometer with 600 nm and/or NIR (Near-infrared) probe. pH is maintained between 4-7 using a 20% phosphoric acid and 0.1 N KOH, as acid and base solutions respectively. Cells in the fermenter are maintained in logarithmic growth phase through continuous addition of fresh medium to the fermenter. A cell recycle system comprised of a 0.2 micron hollow fiber is used to remove the spent medium while maintaining the cells in the fermenter. Medium retention time varies over the length of the fermentation run ranging from a steady state at the start of the process to ≤12 hours at the end of the run. FIG. 1 illustrates bioreactor design, and microbial analysis of the fermentate is performed as described in Example 1.

### Example 3

### Production of Plant/Soil Beneficial Microbe-Free Tea

As described in FIG. 1, the spent medium from the fermentation is removed from the cell recycle system using a 0.2 micron hollow fiber. This tea contains metabolites secreted by the bacteria during fermentation as well as fermentation byproducts beneficial to plants and/or soil microbiomes as described in Table 1. The tea is used crude (i.e. bottled directly) or further refined to enrich and/or isolate compounds with desired traits using established separation techniques such as, but not limited to, affinity chromatography and/or size exclusion chromatography and/or solvent extraction or any combination thereof. Additionally, the tea is dried using spray dry methods for example to produce a soluble powder.

### Example 4

### Producing a Complex Consortium Using Multiple Groups of >2 Bacterial Strains

A consortium of microbes is produced using multiple sub-groups of bacteria. These sub-groups are determined based co-cultivation compatibility studies. Once selected, each group is grown in co-culture to high cell densities based on the process described in Example 1. In this instance, a set of anaerobic microbes composed of but not limited to bacteria from the genera Clostridium and/or Lactobacillus and/or Enterobacter is co-cultivated under anaerobic conditions using the process as described in Example 2. In parallel and in another fermentation vessel, bacteria such as but not limited to the genera Bacillus and/or pseudomonas, and/or Streptomyces and/or Azotobacter are co-cultivated under aerobic conditions as described in Example 1. Upon termination of the fermentation process, the two groups are combined either directly (i.e. in liquid form) or after stabilization such as drying (freeze drying or spray drying) to form the final product. The 1:1 combination varies such as but not limited to 1:2, 2:1 (aerobes: anaerobes) as dictated by efficacy studies in plants. Mixing occurs immediately prior to use or at the time of manufacturing.

In some instances, more than one (1) anaerobic subgroup and/or more than one (1) aerobic subgroup are combined to form the final consortium.

### Example 5

### Conventional Volumetric Seed Train vs Exemplary Seed Train Alternative

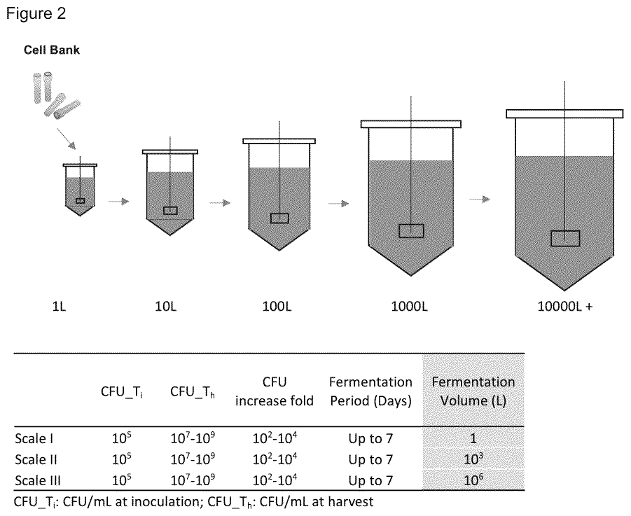

In a conventional volumetric seed train (FIG. 2), one can expect to use a scale-up factor of 10 to 10³. Using the latter, it takes up to 21 days to achieve the final production scale. Under conventional fermentation conditions, bacteria concentrations achieved range between 10⁷ to 10⁹ CFU/mL as evident by the current products on the market as described in Table 2.

TABLE 2
Example of CFU claimed in current microbial products on the market.

| | | CFU/mL |
|---|---|---|
| Product name | Manufacturer | claimed on label |
| Dyna-Start ® X2 | Loveland | 2.0E+09 |
| Nodulator ® Inoculant | BASF Corp. | 1.0E+09 |
| N-TEXX ® | CXi | 2.0E+06 |
| Soil Endhance ® | AgroScience Solutions | 6.6E+07 |
| Quantum-VSC ™ | Ecological Laboratories Inc. | 9.0E+06 |
| Quantum-Organic light ™ | Ecological Laboratories Inc. | 1.0E+06 |
| QuickRoots ® WP | Acceleron BioAg | 3.3E+08 |
| TerraGrow ® | BioSafe Systems | 3.0E+08 |
| Vault ® | BASF Corp | 2.0E+09 |
| | Average: | 6.34E+08 CFU/mL |

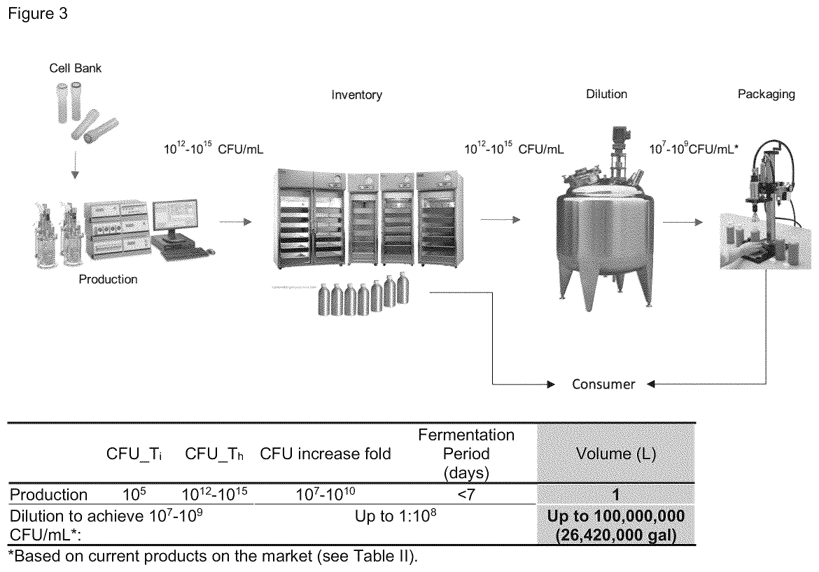

The alternative production process presented in Example 1 conservatively can achieve cell densities >10¹² CFU/mL in 1 L fermenters under 7 days. This constitutes at least 1,000-fold economy of scale and cuts production time by two thirds over the conventional seed trains. Additionally, savings in manufacturing footprint and process control (especially sterility) are anticipated. FIG. 3 illustrates the manufacturing process from inception to the consumer. In one instance, the production of the minimum-scale high-density fermentation is packaged in aliquots that are subsequently diluted to meet current market CFU norms of 10⁷-10⁹ CFU/mL (Table 2). Alternatively, concentrated product is directly sold to consumers for on-site dilution based on scale and needs.

### Example 6

### Flexibility of the Volumetric Seed-Train Alternative Presented Herein

As mentioned previously, the small footprint and high-density fermentation process described is extremely flexible and easily adaptable to various geographies and regulatory challenges. The small manufacturing footprint is easily customizable to meet local and budget needs.

In one instance, a defined or undefined microbial consortium derived from either crude soil, compost, agriculture waste or other source is used to ferment different infusions separately yet in parallel, producing different microbial products which is combined at various ratios. Infusions are derived from agriculture byproducts such as plant waste and/or composts or from the byproducts of beverage/food industry. Each substrate (infusion) supports the enrichment of microbial population subsets present the original consortium (inoculum) based on the affinity each microbe in the mix has to the different nutrients in the infusions.

Alternatively, a particular infusion is fermented under varying conditions to produce different metabolites and fermentation products. These conditions such as but not limited to oxygen availability, pH, salinity, osmolarity, temperature and combinations thereof trigger the differential multiplication of subgroups of microbes from the inoculum leading to different products with different practical benefits for plants, soils or other applications. Alternatively, the metabolic profile of the same set of microbes will change as they ferment the infusion under different conditions.

1. A method of preparing a high density culture of bacteria comprising the steps of:
i) providing a bioreactor, wherein the bioreactor comprises a fermentation tank, a perfusion system, a medium, and a cell separator;
ii) preparing an inoculum, wherein the inoculum comprises a bacteria consortia, wherein the bacteria consortia comprises at least two bacteria species;
iii) inoculating the fermentation tank with the inoculum to form a fermentation culture; and
iv) growing the bacteria consortia until it has reached a target density, wherein spent medium in the fermentation tank is removed and fresh medium is added to the fermentation tank by the perfusion system while the bacteria consortia is growing, wherein the target density is at least about 10 12 CFU/mL.

2. The method of claim 1, further comprising monitoring the fermentation culture while the bacteria consortia is growing, wherein the fermentation culture is monitored for exponential growth of the bacteria consortia.

3. The method of claim 2, wherein the monitoring is performed by a sensor.

4. The method of claim 3, wherein the sensor is an Optical Density (OD) sensor, a pH probe, a dissolved oxygen probe, or a foam sensor.

5. The method of claim 1, further comprising v) harvesting the spent medium into a package.

6. The method of claim 1, further comprising v) harvesting the bacteria consortia into a package.

7. The method of claim 6, further comprising vi) diluting the harvested content so that the density of the bacteria consortia in the package is about 10 8 CFU/mL.

8. The method of claim 5, wherein the harvesting occurs within about 7 days of inoculating the fermentation tank.

9. The method of claim 6, further comprising inoculating a second bioreactor with the harvested bacteria consortia.

10. A method of preparing a high density culture of bacteria comprising the steps of:
i) providing a bioreactor, wherein the bioreactor comprises a fermentation tank, a perfusion system, a medium, and a cell separator;
ii) preparing an inoculum, wherein the inoculum comprises a bacteria consortia, wherein the bacteria consortia comprises at least two aerobic bacteria species;
iii) inoculating the fermentation tank with the inoculum to form a fermentation culture; and
iv) growing the bacteria consortia until it has reached a target density, wherein spent medium in the fermentation tank is removed and fresh medium is added to the fermentation tank by the perfusion system while the bacteria consortia is growing, wherein the target density is at least about 10¹² CFU/mL.

11. The method of claim 10, further comprising v) harvesting the bacteria consortia.

12. The method of claim 11, further comprising vi) combining the harvested bacteria consortia with an anaerobic bacteria consortia.

13. A high density bacterial culture process that includes two or more of the following steps:
i) inoculating and growing individual fresh inoculum cultures comprising a target bacterial strain to late exponential phase; (to be grown to high density in a final fermentation vessel, where said inoculum cultures are seeded using freshly cultured isolates or subcultures of preserved stocks (cryopreserved, frozen, plated, refrigerated or otherwise dormant but viable stocks);
ii) separately combining a pre-processed organic slurry of plant material with a sufficient volume of sterile diluent to make a tea slurry;
iii) allowing nutrients to effuse out of the organic slurry into the liquid tea component of the tea slurry;
iv) harvesting the liquid tea from the tea slurry;
v) eliminating plant-sourced microbes from the tea to make a sterile growth infusion; (using a micro-filtration, pasteurization or other sterilization methodology);
vi) setting up a sterile fermentation vessel with all required components to promote growth of the target bacteria; (comprising one or more of the following: buffers, anti-foaming agents, monitoring probes, recycling and aeration feeds, stirring mechanisms, and any appropriate growth components);
vii) adding sufficient media, sterile growth infusion, and inoculum cultures to the fermentation vessel making a fermentation culture;
viii) growing the fermentation culture to late exponential phase; comprising (i) an appropriate amount of culture medium designed for desired growth of the target bacterium in the fermentation vessel; (ii) a desired amount of nutrient rich growth infusion(s) (ratio(s) can be varied depending on desired results) at a pre-determined setup infusion-media ratio; (iii) inoculate the fermentation vessel with a desired amount of fresh bacterial inoculum culture (Step a);
ix) initiating a media replacement regimen to replace spent media generated in the active fermentation culture with fresh media and sterile growth infusion;(whereby the spent culture medium of the fermentation culture is gradually, and continuously harvested through a filtration system, leaving all target bacteria in the fermenter, while fresh growth infusion-media (which may have the same or a different ratio than the setup infusion-media ratio) is pumped into the fermentation vessel at a rate that may be the same as or different from the rate of spent culture medium harvesting);
x) continuously harvesting the spent medium from the fermenter;
xi) monitoring the fermentation culture for exponential growth of the target bacteria;
xii) terminating the fermentation culture process when the target bacterial biomass is achieved; and
xiii) harvesting the desired target bacterial cells from the fermenter.

14. A method of preparing a medium for the high density culture of bacteria comprising the steps of:
i) combining a pre-processed organic slurry of plant material with a sterile diluent to make a tea slurry, wherein the tea slurry comprises a liquid tea component;
ii) allowing nutrients to effuse out of the pre-processed organic slurry into the liquid tea component of the tea slurry;
iii) harvesting the liquid tea from the tea slurry; and
iv) eliminating the plant-sourced microbes from the tea to make a sterile growth infusion, wherein a medium for the high density culture of bacteria comprises the sterile growth infusion.

15. The method of claim 14, wherein the plant sourced microbes are eliminated using micro-filtration or pasteurization.

16. A high density culture of bacteria, produced by the method of claim 10.

17. A high density culture of bacteria, produced by the method of claim 1.

18. A high density culture of bacteria, produced by the method of claim 13.

19. A high density culture of bacteria, produced by the method of claim 14.
